(19) United States
(12) Patent Application Publication
(10) Pub. No.: US 2004/0224877 A1
(43) Pub. Date: Nov. 11, 2004

(54) Method and Composition for the Treatment of Angiogenesis

(76) Inventors: Steven Pirie-Shepherd, Cardiff, CA (US); Oliver Kisker, Rossdorf (DE); Shinya Onizuka, Nagasaki (JP); Rahul Ray, Wayland, MA (US); Swamy Narasimha, Providence, RI (US); M. Judah Folkman, Brookline, MA (US)

Correspondence Address: David S Resnick, Nixon Peabody, 101 Federal Street, Boston, MA, 02110, US

(21) Appl. No.: 10/275,414

(22) PCT Filed: May 9, 2001
(86) PCT No.: PCT/US01/15107

Related U.S. Application Data
(60) Provisional application No. 60/203,150, filed on May 9, 2000.

(51) Int. Cl. A61K 38/17

(52) U.S. Cl. 514/12

(57) ABSTRACT

We have now discovered that partially deglycosylated vitamin D binding protein (DBP-maf) is anti-tumorigenic not wholly via an immune mechanism but in part via an antiangiogenic mechanism. Accordingly, the present invention relates to use of DBP-maf in the treatment of diseases associated with increased or abnormal angiogenesis and/or endothelial cell differentiation. Preferably, the DBP-maf is administered to the patient by sustained release or in pulses. Pulse therapy is not a form of discontinuous administration of the same account of a composition over time, but comprises administration of the same dose of the composition at a reduced frequency or administration of reduced doses.

This invention was made in part with U.S. Government support under NIDDKD Grant No. DK 44337. The U.S. Government has certain rights in this application.

### FIELD OF THE INVENTION

The present invention provides for a novel pharmaceutical composition, and method of use thereof for treatment of diseases or disorders involving angiogenesis.

### BACKGROUND OF THE INVENTION

Blood vessels are the means by which oxygen and nutrients are supplied to living tissues and waste products are removed from living tissue. Angiogenesis refers to the process by which new blood vessels are formed. (31). Thus, where appropriate, angiogenesis is a critical biological process. It is essential in reproduction, development and wound repair. However, inappropriate angiogenesis can have severe negative consequences. For example, it is only after many solid tumors are vascularized as a result of angiogenesis that the tumors have a sufficient supply of oxygen and nutrients that permit it to grow rapidly and metastasize. Because maintaining the rate of angiogenesis in its proper equilibrium is so critical to a range of functions, it must be carefully regulated in order to maintain health. The angiogenesis process is believed to begin with the degradation of the basement membrane by proteases secreted from endothelial cells (EC) activated by mitogens such as vascular endothelial growth factor (VEGF) and basic fibroblast growth factor (bFGF). The cells migrate and proliferate, leading to the formation of solid endothelial cell sprouts into the stromal space, then, vascular loops are formed and capillary tubes develop with formation of tight junctions and deposition of new basement membrane.

In adults, the proliferation rate of endothelial cells is typically low compared to other cell types in the body. The turnover time of these cells can exceed one thousand days. Physiological exceptions in which angiogenesis results in rapid proliferation typically occurs under tight regulation, such as found in the female reproduction system and during wound healing.

The rate of angiogenesis involves a change in the local equilibrium between positive and negative regulators of the growth of microvessels. The therapeutic implications of angiogenic growth factors were first described by Folkman and colleagues over two decades ago (32). Abnormal angiogenesis occurs when the body loses at least some control of angiogenesis, resulting in either excessive or insufficient blood vessel growth. For instance, conditions such as ulcers, strokes, and heart attacks may result from the absence of angiogenesis normally required for natural healing. In contrast, excessive blood vessel proliferation can result in tumor growth, tumor spread, blindness, psoriasis and rheumatoid arthritis.

Thus, there are instances where a greater degree of angiogenesis is desirable—increasing blood circulation, wound healing, and ulcer healing. For example, recent investigations have established the feasibility of using recombinant angiogenic growth factors, such as fibroblast growth factor (FGF) family (33, 34), endothelial cell growth factor (ECGF) (35), and more recently, vascular endothelial growth factor (VEGF) to expedite and/or augment collateral artery development in animal models of myocardial and hindlimb ischeria (36, 37).

Conversely, there are instances, where inhibition of angiogenesis is desirable. For example, many diseases are driven by persistent unregulated angiogenesis, also sometimes referred to as “neovascularization.” In arthritis, new capillary blood vessels invade the joint and destroy cartilage. In diabetes, new capillaries invade the vitreous, bleed, and cause blindness. Ocular neovascularization is the most common cause of blindness. Tumor growth and metastasis are angiogenesis-dependent. A tumor must continuously stimulate the growth of new capillary blood vessels for the tumor itself to grow.

The current treatment of these diseases is inadequate. Agents which prevent continued angiogenesis, e.g, drugs (TNP-470), monoclonal antibodies, antisense nucleic acids and proteins (angioslatin (5), endostatin (6) and antiangiogenic ATIII (7)) are currently being tested. (38, 39, 40). Although preliminary results with the antiangiogenic proteins are promising, they are relatively large in size and their difficult to use and produce. Moreover, proteins are subject to enzymatic degradation. Thus, new agents that inhibit angiogenesis are needed. New antiangeogenic angents that show improvement in size, ease of production, stability and/or potency would be desirable.

Vitamin D Binding protein (DBP) is a multi-domain protein that binds vitamin D catabolites and metabolites at its aminoterminal domain, and actin at it's carboyterminal domain (9). Further, the carboxyterminal domain contains an O-linked glycosylation site on a threonine residue in human DBP (10). This site is occupied with a mucin-type trisaccharide, consisting of N-acetylgalactosamine with a dibranched galactose and sialic acid. Sequential removal of the terminal sialic acid and galactose results in a molecule with core N-acetylgalactosamine attached to the threonine residue (10). Such selective deglycosylation occurs naturally as part of the immune response. The resultant molecule is a potent activator of macrophage, and is termed DBP-maf (macrophages activating factor).

Previous data has shown that DBP-maf, generated specifically by selective in vitro deglycosylation, has a role to play in the treatment of an Ehrlich ascites tumor in a mouse model (11, 12). Further administration of DBP-maf, as an adjuvant immunotherapy to photodynamic therapy of cancer (13) had a synergistic effect on tumor cures using a squamous cell carcinoma model in mice. In both tumor models, it was hypothesised that DBP-maf elicited it's effect by an inflammatory process, e.g., activating macrophages which then directly attacked the tumor cells.

### SUMMARY OF THE INVENTION

We have now discovered that partially deglycosylated vitamin D binding protein (DBP-maf) is anti-tumorigenic not wholly via an immune mechanism but in part via an antiangiogenic mechanism. Accordingly, the present invention relates to use of DBP-maf in the treatment of diseases associated with increased or abnormal angiogenesis and/or endothelial cell differentiation. Preferably, the DBP-maf is administered to the patient by sustained release or in pulses. Pulse therapy is not a form of discontinuous administration of the same amount of a composition over time, but comprises administration of the same dose of the composition at a reduced frequency or administration of reduced doses.

Sustained release or pulse administration are particularly preferred when the angiogenesis is associated with tumor growth as it leads to regression of blood vessels feeding the tumor and ultimately to regression of the tumor itself.

The present invention also relates to a method of inhibiting angiogenesis at a site of a tumor in an immunocompromised mammal by administration of an effective amount of DBP-maf to inhibit angiogenesis at the tumor site in the immunocomprised mammal.

DBP-maf will bind 25 (OH) vitamin D and its metabolites 1,25 (OH)₂ vitamin D and other catabolites including calcitriol. Calcitriol is currently in clinical trials as a drug therapy for various cancers including prostate cancer. One of the disadvantages of using Calcitriol in vivo is side-effects such as hypercalcemia at doses above physiological levels. While not wishing to be bound by theory, we believe that Calcitriol bound to DBP-maf will target the proliferating endothelium found in a growing tumor thus attacking the tumor on two fronts. This approach further allows for use of lower doses of Calcitriol and DBP-maf and thus avoids issues of drug toxicity and acquired drug resistance.

Accordingly, the present invention also provides a composition comprising partially deglycosylated Vitamin D binding protein (DBP-maf) and 1,25(OH)₂ Vitamin D (calcitriol) or a derivative thereof.

This composition is useful in a method for treating hormone dependent cancer. The method comprises administering to a host having a hormone dependent cancer an effective amount of the composition comprising deglycosylated vitamin D binding protein (DBP-maf) and 1,25(OH)₂ Vitamin D or a derivative thereof. Preferred hormone dependent cancers include breast and prostate.

Other aspects of the invention are disclosed infra.

### BRIEF DESCRIPTION OF THE DRAWINGS

FIGS. 1A-C show purification of anti-endothelial factor from human pancreatic cancer cell, BxPC3. BxPC3 conditioned media (1 L conditioned for 48 hours at 37° C. in the presence of 5% FCS) was applied to an HiTrap (5 mL) heparin-Sepharose column (Pharmacia) equilibrated in 50 mM Tris pH 7.4. The unbound material contained anti-endothelial activity, as assayed by an endothelial cell proliferation assay. This activity (•-•) was further purified using Q-Sepharose (FIG. 1A) and monoQ anion exchange chromatography (FIG. 1B). Note that control media containing 5% fetal calf serum exhibited no anti-endothelial activity (∘-∘) when subjected to the fractionation protocol. The fraction exhibiting anti-endothelial activity (•-•) eluting from monoQ at ˜0.2 M NaCl (from a continuous gradient of NaCl) contained two bands (FIG. 1C). These bands were very similar in migration and pI. They were resolved from each other using C4 RP-HPLC, and sequence analysis, coupled with mass spectrometry analysis, determined that they were BSA and a protein with 90% homology to human vitamin D binding protein within the aminoterminal 10 residues. We therefore assigned an identity of bovine vitamin D binding protein to this band.

FIG. 2 is a graph showing macrophage activation by DBP-maf. Various DBP preparation were generated and tested for their ability to activate macrophages, as measured by a superoxide production assay/human DBP and bovine DBP were unable to activate macrophages. Human DBP-maf and bovine DBP-maf, generated by selective and specific deglycosylation, were able to activate macrophages. Bovine DBP purified from BxPC3 conditioned media, was also able to activate macrophages.

FIG. 3A is a graph showing treatment of BxPC3 human pancreatic tumor with DBP-maf. BxPC3 cells were implanted s.c. onto the flank of a SCID mouse. Once the tumor had attained a volume of 100 mm³ treatment was started. Animals were given 4 ng/Kg/day of DBP-maf (∘-∘). Therapy was administered i.p. Control animals ( - ) received saline injections only. n=3

FIG. 3B is a graph showing treatment of SU88.86 human pancreatic tumor with DBP-maf. SU88.86 cells were implanted s.c. onto the flank of a SCID mouse. Once the tumor had attained a volume of 100 mm³ treatment was started. Animals were given 4 ng/Kg/day of DBP-maf (∘-∘). Therapy was administered i.p. Control animals (●-●) received saline injections only. n=3

FIG. 3C is a graph showing treatment of Lewis Lung Carcinoma with DBP-maf. Lewis lung carcinoma cells were implanted s.c. onto the flank of a C57/B16 mouse. Once the tumor had attained a volume of 100 mm³ treatment was started. Animals were given 4 ng/Kg/day of DBP-maf (∘-∘). Therapy was administered i.p. Control animals (●-●) received an equivalent dose of DBP expressed in E coli (and hence lacking any O-linked carbohydrate). n=3

FIG. 4A is a graph showing treatment of BxPC3 human pancreatic cancer with increasing doses of DBP-maf. BxPC3 cells were implanted s.c. onto the flank of a SCID mouse. Once the tumor had attained a volume of 100 mm³ treatment was started. Animals received i.p injections of DBP-maf at 4 ng/Kg/4 days (∘-∘), 4 ng/Kg/day (▪-▪), or 4 μg/Kg/day (□-□). Control animals received 4 μg/Kg/day of E. coli expressed human DBP (●-●). n=5.

FIG. 4B is a photograph showing treatment of BxPC3 human pancreatic cancer with increasing doses of DBP-maf. Tumors excised at the end of the experiment. Top row shows tumors excised from animals that received 4 μg/Kg/day of E coli expressed human DBP (c.f. FIG. 4A ●-●). Bottom row shows tumors excised from animals that received 4 μg/Kg/day of human DBP-maf (c.f. FIG. 4A □-□).

FIGS. 5A-5H are photographs of histological examination of control and treated tumors. Control untreated BxPC3 tumor (FIG. 5A) and DBP-maf treated tumors (FIG. 5B) were stained with H&E. Sections were stained with an antibody directed against CD31 (PECAM), note that in control tumor (FIG. 5C) the tumor is vascularized (arrow), whereas in treated tumor tissue (FIG. 5D), the tumor cells are poorly vascularized, although some vascularization is seen in the connective tissue adjacent to the tumor (arrow). Staining for the antigen CD45 revealed few cells of monocyte lineage in control tumors (FIG. 5E) (arrow), whereas heavy infiltration of monocyte-like cells into the treated tumor (FIG. 5F) was apparent (arrow). Staining for leucocyte common antigen (LCA) also showed heavy infiltration of DBP-maf treated tumors by cells of leucocyte origin (FIG. 5G). Control tumors stained poorly for this antigen (FIG. 5H).

FIGS. 6A and 6B show inhibition of angiogenesis in the CAM by human DBP and DBP-maf Human vitamin D binding protein (DBP) was purified from human plasma using a combination of vitamin-D-Sepharose affinity chromatography and hydroxyapatite chromatography as described infra. The purified protein was then converted to DBP-maf by galactosidase and sialidase as described (see Materials and Methods). DBP-maf inhibited angiogenesis in the CAM assay at a dose of 500 ng/egg (FIG. 6A), and 5 μg/egg (FIG. 6B).

### DETAILED DESCRIPTION OF THE INVENTION

Angiogenesis is the formation of new blood vessels. Angiogenesis occurs in a variety of physiologic and pathological processes such as embryonic growth, ovulation, cyclical development of the uterine endometrium, wound healing, inflammation, diabetic retinopathy and tumor growth.

The present invention relates to inhibition of angiogenesis by DBP-maf and by analogs, derivatives and fragments of DBP-maf or mixtures thereof. DBP-maf, analogs, derivatives and fragments thereof are useful in treating angiogenic diseases in mammals so afflicted by such a disease.

Angiogenic diseases amenable to treatment using DBP-maf and analogs, derivatives and fragments thereof include but are not limited to diabetic retinopathy, retrolental fibroplasia, trachoma, neovascular glaucoma, psoriases, angio-fibromas, immune and non-immune inflammation, capillary formation within atherosclerotic plaques, hemangiomas, excessive wound repair, solid tumors, Kaposi's sarcoma and the like.

DBP-maf and analogs, derivatives and fragments thereof inhibit neovascularization at a site. DBP-maf, analogs, derivatives and fragments thereof, are effective directly or indirectly in inhibiting the function of inducers of angiogenesis. Such inducers of angiogenesis amenable to regulation by DBP-maf include but are not limited to basic fibroblast growth factor (bFGF), vascular endothelial growth factor (VEGF), hepatocyte growth factor (i.e. scatter factor), IL-8 and the like.

The present invention demonstrates that DBP-maf inhibits angiogenesis at a site of tumor growth in immunocompromised or immunodeficient mammals as demonstrated in immunocompromised SCID mice. These mice are deficient in B- and T-lymphocytes. Therefore, the inhibitory effect of DBP-maf on tumor growth in the immunocompromise mammal is not dependent on B- or T-lymphocytes. These findings are in sharp contrast to those in Yamamoto et al (11) and Koga et al (12) who teach that the DBP-maf antitumor response is an immune mediated mechanism.

DBP-maf and analogs, derivatives and fragments thereof are useful therapeutics for inhibiting angiogenesis at a site of tumorigenesis or tumor growth in an immunocompromised mammal, preferably a T-lymphocyte deficient and/or B-lymphocyte deficient mammal. Immunocompromised mammals amenable to treatment with DBP-maf include but are not limited to mammals including humans with genetic or acquired-immunodeficiency syndromes, mammals on immunosuppressive agents such as mammals pre- and post-transplantation, mammals with autoimmune diseases, mammals with viral infections and the like who are T-lymphocyte deficient and/or B-lymphocyte deficient. Treatment of mammals with immunosuppressive agents such as cyclosporin, anti-T or anti-B cell antibodies, prednisone, irradiation, and the like for prolonged periods of time place the mammal at risk of development of tumors. DBP-maf of the present invention is useful in inhibiting the angiogenesis at the site of tumorigenesis or tumor growth in these immunosuppressed mammals and in turn preventing or inhibiting tumor growth.

Patients with congenital immunodeficiency syndromes who may benefit from treatment with DBP-maf include those affected by Severe Combined Imunodeficiency, Adenosine Deaminase Deficiency, DiGeorge Syndrome, Immunodeficiency with Thymoma, Immunodeficiency following hereditary defective response to Epstein-Barr virus (Purtilo Syndrome), Ataxia-telangiectasia, Wiscott-Aldrich Syndrome, or Intestinal Lymphangiectasia.

Patients with acquired immunodeficiency syndromes who may benefit from treatment with DBP-maf can be distinguished in groups depending on the cause of the immunosuppression.

A. The immunosuppression may be the result of treatment with various classes of agents, including, but not limited to, a) corticosteroids; b) cytotoxic drugs, a family of drugs that includes alkylating agents (for example cyclophosphamide, chlorambucil and nitrogen mustard), purine analogs (for example azathioprine, mercaptopurine, and thioguanine) and folic acid antagonists (for instance methotrexate, and other drugs such as 5-fluorouracil, vinca alkaloid, hydroxyurea, and antibiotic immunosuppressants; c) ionizing irradiation; d) antibody therapy with anti-lymphocyte serum or monoclonal antibodies; immunosuppressive agents such as cyclosporin A; and immunosuppressive cytokines, including interferon alpha, TGF-beta, and Interleukin 10.

B. The immunosuppression may be the consequence of an acquired disease/syndrome. This includes, but is not limited to AIDS, Kaposi sarcoma, lymphomas and other malignancies in immunopriviledged sites such as the central nervous syndrome, recipients of bone marrow, stem cells, or solid organ transplants, Hodgkin's lymphoma, HTLV-1 associated T cell leukemias/lymphomas, Lupus Erythematosus, Rheumatoid arthritis, systemic vasculitis, erythema nodosum, scleroderma, Sjogren's syndrome, sarcoidosis, and primary biliary cirrhosis.

The presence of T and/or B cell inununodeficiency represents no contraindication for use of DBP-maf because DBP-maf was shown not to require T cells or B-cells or their products to inhibit angiogenesis. Indeed the antiangiogenic effect of DBP-maf was reproducibly demonstrated in immunecomprimised mice where no significant numbers of T or B cells and T or B cell functions were present. Further, in vitro endothelial cell proliferation assays and CAM assays demonstrate that the antiangiogenic property of DBP-maf is not by an immune mechanism.

Thus, in contrast to the observations of Yamamoto et al (11) and Koga et al (12), the present invention encompasses all patients who might benefit from treatment with DBP-maf and exhibit various levels of congenital or acquired T and/or B cell immunodeficiency.

The present invention encompasses combination therapy in which DBP-maf is administered in conjunction with another anti-angiogenic agent for inhibiting angiogenesis. Other anti-angiogenic agents that may be administered in conjunction with DBP-maf include but are not limited to angiostatin, endostatin, PF4, IFN-γ, TNP470, thrombospondin and the like.

Combination therapy also encompasses the use of DBP-maf in combination with a chemotherapeutic agent such as Taxol, cyclophosphamide, cisplatin, gancyclovir and the like. Such a therapy is particularly useful in situations in which the mammal to be treated has a large preexisting tumor mass which is well vascularized. The chemotherapeutic agent serves to reduce the tumor mass and the DBP-maf prevents or inhibits neovascularization within or surrounding the tumor mass. The chemotherapeutic agent may also be administered at lower doses than normally used and at such doses may act as an antiangiogenic agent.

DBP-maf for use in the present invention may be obtained from natural, recombinant or synthetic sources. It is important to note that partial glycosylation of this protein is a necessary requirement for its antiangiogenic function. DBP-maf may be obtained by the methods set forth in U.S. Pat. No. 5,177,001. In the case of natural sources, DBP-maf may be purified and isolated from any mammalian species, preferably from human sources. Analogs according to the invention may include peptides with conservative amino acid substitutions or non-conservative amino acid substitutions, deletions or insertions which do not lessen the anti-angiogenic activity of the DBP-maf. The invention also includes DBP-maf coupled to carbohydrates such as PEG or protein carriers as long as the analog retains its anti-angiogenic function. Synthetic fragments of DBP-maf may be made by standard methods of peptide synthesis as are known in the art.

DBP-maf of the present invention is usefull in inhibiting the angiogenic function of target cells both in vitro and in vivo. DBP-maf of the present invention is particularly useful in inhibiting the angiogenic function of endothelial cells both in vitro and in vivo. Of particular interest is the prevention or inhibition of endothelial cell differentiation into capillary structures. The endothelial cells amenable to inhibition by DBP-maf are present at several sites in a mammal and include but are not limited to dermis, epidermis, endometrium, retina, surgical sites, gastrointestinal tract, liver, kidney, reproductive system, skin, bone, muscle, endocrine system, brain, lymphoid system, central nervous system, respiratory system, umbilical cord, breast tissue, urinary tract and the like. The method of treatment of the present invention using DBP-maf is particularly useful in preventing or inhibiting angiogenesis by endothelial cells at sites of inflammation and tumorigenesis.

Angiogenesis associated with autoimmune diseases may be treated using DBP-maf. The autoimmune diseases include but are not limited to rheumatoid arthritis, systemic lupus erythematosus, thyroiditis, Goodpasture's syndrome, systemic vasculitis, scleroderma, Sjogren's syndrome, sarcoidosis, primary biliary cirrhosis and the like.

Angiogenesis associated with wound repair may also be treated using DBP-maf. Excessive scarring resulting from excess angiogenesis often occurs at sites of skin trauma or surgical sites. Administration of DBP-maf at the site is useful in preventing or inhibiting angiogenesis at the site to eliminate or lessen the scarring.

DBP-maf is also useful in methods of inhibiting angiogenesis at a site of tumorigenesis in a mammal. DBP-maf administered at such sites prevents or inhibits blood vessel formation at the site thereby inhibiting the development and growth of the tumor. Tumors which may be prevented or inhibited by preventing or inhibiting angiogenesis with DBP-maf include but are not limited to melanoma, metastases, adenocarcinoma, sarcomas, thymoma, lymphoma, lung tumors, liver tumors, colon tumors, kidney tumors, non-Hodgkins lymphoma, Hodgkins lymphoma, leukemias, uterine tumors, breast tumors, prostate tumors, renal tumors, ovarian tumors, pancreatic tumors, brain tumors, testicular tumors, bone tumors, muscle tumors, tumors of the placenta, gastric tumors and the like.

Preferably, the DBP-maf is administered to the patient by sustained release or in pulses. Pulse therapy is not a form of discontinuous administration of the same amount of a composition over time, but comprises administration of the same dose of the composition at a reduced frequency or administration of reduced doses. Sustained release or pulse administration are particularly preferred when the angiogenesis is associated with tumor growth as it leads to regression of blood vessels feeding the tumor and ultimately to regression of the tumor itself. Each pulse dose can be reduced and the total amount of drug administered over the course of treatment to the patient is minimized.

Individual pulses can be delivered to the patient continuously over a period of several hours, such as about 2, 4, 6, 8, 10, 12, 14 or 16 hours, or several days, such as 2, 3, 4, 5, 6, or 7 days, preferably from about 1 hour to about 24 hours and more preferably from about 3 hours to about 9 hours.

The interval between pulses or the interval of no delivery is greater than 24 hours and preferably greater than 48 hours, and can be for even longer such as for 3, 4, 5, 6, 7, 8, 9 or 10 days, two, three or four weeks or even longer. As the results achieved may be surprising, the interval between pulses, when necessary, can be determined by one of ordinary skill in the art. Often, the interval between pulses can be calculated by administering another dose of the composition when the composition or the active component of the composition is no longer detectable in the patient prior to delivery of the next pulse. Intervals can also be calculated from the in vivo half-life of the composition. Intervals may be calculated as greater than the in vivo half-life, or 2, 3, 4, 5 and even 10 times greater the composition half-life.

The number of pulses in a single therapeutic regimen may be as little as two, but is typically from about 5 to 10, 10 to 20, 15 to 30 or more. In fact, patients can receive drugs for life according to the methods of this invention without the problems and inconveniences associated with current therapies. Compositions can be administered by most any means, but are preferable delivered to the patient as an injection (e.g. intravenous, subcutaneous, intraarterial), infusion or instillation. Various methods and apparatus for pulsing compositions by infusion or other forms of delivery to the patient are disclosed in U.S. Pat. Nos. 4,747,825; 4,723,958; 4,948,592; 4,965,251 and 5,403,590.

Compositions comprising DBP-maf can also be administered by sustained release. Sustained release may be accomplished by means of an osmotic pump. Preferably the DBP-maf is administered over a period of several days, such as 2, 3, 4, 5, 6 or 7 days.

In the method of treatment, the administration of DBP-maf, analogs, derivatives or fragments thereof may be for either “prophylactic” or “therapeutic” purpose. When provided prophylactically, the DBP-maf is provided in advance of any symptom. The prophylactic administration of the DBP-maf serves to prevent or inhibit any angiogenesis at a site. When provided therapeutically, the DBP-maf is provided at (or after) the onset of a symptom or indication of angiogenesis. Thus, DBP-maf may be provided either prior to the anticipated angiogenesis at a site or after the angiogenesis has begun at a site.

The term “unit dose” as it pertains to an inoculum refers to physically discrete units suitable as unitary dosages for mammals, each unit containing a predetermined quantity of DBP-maf, analogs, derivatives or fragments thereof calculated to produce the desired inhibitory effect in association with a diluent. The specifications for the novel unit dose of an inoculum of this invention are dictated by and are dependent upon the unique characteristics of the DBP-maf and the particular effect to be achieved.

The inoculum is typically prepared as a solution in tolerable (acceptable) diluent such as saline, phosphate-buffered saline or other physiologically tolerable diluent and the like to form an aqueous pharmaceutical composition. In addition, the DBP-maf, analogs, derivatives or fragments thereof may be formulated in solid form and lyophilized form and redissolved or suspended prior to use.

The route of administration may be intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, intratumor and the like.

Administration may be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may by through nasal sprays, for example, or using suppositories. For oral administration, DBP-maf is formulated into conventional oral administration forms such as capsules, tablets and tonics.

For topical administration, DBP-maf is formulated into ointments, salves, gels, or creams, as is generally known in the art.

In providing a mammal with the DBP-maf, preferably a human, the dosage of administered DBP-maf will vary depending upon such factors as the mammal's age, weight, height, sex, general medical condition, previous medical history, disease progression, tumor burden, route of administration, formulation and the like.

In general, it is desirable to provide the recipient with a dosage of DBP-maf of at least about 0.1 μg/kg, preferably at least about 25 μg/kg, more preferably at least about 50 μg/kg or higher. A range of from about 1 μg/kg to about 100 μg/kg is preferred although a lower or higher dose may be administered. The dose provides an effective antiangiogenic serum or tissue level of DBP-maf. The dose is administered at least once and may be provided as a bolus, a continuous administration or sustained release. Multiple administration over a period of weeks or months may be preferable. It may also be preferable to administer DBP-maf at least once/week and even more frequent administrations (e.g. daily) may yield yet more preferable results. Subsequent doses may be administered as indicated.

It may be appropriate to modify DBP-maf by attaching compounds, such as but not limited to PEG, to prolong its half-life without diminishing its anti-angiogenic property.

For combination therapy, the dose of DBP-maf may be administered prior to, concurrently, or after administration of a second anti-angiogenic agent or chemotherapeutic agent.

The dose of a second anti-angiogenic agent or chemotherapeutic agent for administration in combination with DBP-maf are doses routinely used in the art.

The present invention also encompasses a pharmaceutical composition capable of inhibiting angiogenesis which comprises DBP-maf, analogs, derivatives or fragments thereof in a pharmaceutically acceptable carrier. The pharmaceutical composition may additionally comprise a second anti-angiogenic agent including but not limited to angiostatin, PF4, IFN-γ, TNP-470, thrombospondin, and the like or mixtures thereof.

In another embodiment, the pharmaceutical composition also includes a chemotherapeutic agent such as taxol, cyclophosphamide, cisplatin, gancyclovir, and the like or mixtures thereof.

The efficacy of treatment may be assessed by various parameters including 1) tumor size reduction, as determined by measurement of cutaneous masses (such as melanoma, Kaposi sarcoma, etc.), X-rays, scans and other means of tumor size evaluation; 2) lack of tumor progression; 3) reduced keloid formation, as determined by measurement and evaluation of superficial lesions; 4) improvement of retinal lesions associated with diabetic retinopathy, as determined by comparative analysis of photographs of the retinal findus and other appropriate methods of evaluation; 5) lack of progression of diabetic retinopathy. Blood pharmacological levels of DBP-maf may be determined by ELISA, capture assays, radioimmunoassays and receptor binding assays and the like.

The methods described herein are useful in screening analogs, derivatives, fragments and chimeric protein of DBP-maf for anti-angiogenic activity.

Additionally, a selectively DBP-maf will bind 25 (OH) vitamin D its metabolites 1,25 (OH)₂ vitamin D and other catabolites including calcitriol. Calcitriol is currently in clinical trials as a drug therapy for various cancers including prostate cancer. One of the disadvantages of using Calcitriol in vivo is side-effects such as hypercalcemia at doses above physiological levels. While not wishing to be bound by theory, we believe that Calcitriol bound to DBP-maf will target the proliferating endothelium found in a growing tumor thus attacking the tumor on two fronts. This approach further allows for use of lower doses of Calcitriol and DBP-maf, and thus avoids issues of drug toxicity and acquired drug resistance.

Accordingly, the present invention also provides a composition comprising partially deglycosylated Vitamin D binding protein (DPB-MAF) and 1,25(OH)₂ Vitamin D (calcitriol) or a derivative thereof. Derivatives of Calcitriol are disclosed in, for example, U.S. Pat. No. 5,976,784.

This composition is useful in a method for treating hormone dependent cancer. The method comprises administering to a host having a hormone dependent cancer an effective amount of the composition comprising deglycosylated vitamin D binding protein (DBP-maf) and 1,25(OH)₂ Vitamin D or a derivative thereof. Preferred hormone dependent cancers include breast and prostate.

The term “pharmaceutically acceptable” refers to compounds and compositions which may be administered to mammals without undue toxicity. Exemplary pharmaceutically acceptable salts include mineral acid salts such as hydrochlorides, hydrobromides, phosphates, sulfates, and the like; and the salts of organic acids such as acetates, propionates, malonates, benzoates, and the like.

The composition of the invention is orally, topically, or by parenteral means, including subcutaneous and intramuscular injection, implantation of sustained release depots, intravenous injection, intranasal administration, and the like. Accordingly, the composition of the invention is preferably administered as a pharmaceutical composition comprising a DBP maf/cal of the invention in combination with a pharmaceutically acceptable carrier. Such compositions may be aqueous solutions, emulsions, creams, ointments, suspensions, gels, liposomal suspensions, and the like. Suitable carriers (excipients) include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol Registered TM, vegetable oils, and the like. One may additionally include suitable preservatives, stabilizers, antioxidants, antimicrobials, and buffering agents, for example, BHA, BHT, citric acid, ascorbic acid, tetracycline, and the like. Cream or ointment bases useful in formulation include lanolin, Silvadene Registered TM (Marion), Aquaphor Registered TM (Duke Laboratories), and the like. Other topical formulations include aerosols, bandages, and other wound dressings. Alternatively one may incorporate or encapsulate the therapeutic compound of the invention in a suitable polymer matrix or membrane, thus providing a sustained-release delivery device suitable for implantation near the site to be treated locally. Other devices include indwelling catheters and devices such as the Alzet Registered TM minipump. Further, one may provide a therapeutic compound of the invention in solid form, especially as a lyophilized powder. Lyophilized formulations typically contain stabilizing and bulking agents, for example human serum albumin, sucrose, mannitol, and the like. A thorough discussion of pharmaceutically acceptable excipients is available in Remington's Pharmaceutical Sciences (Mack Pub. Co.).

The amount of the composition of the invention required to treat a hormone dependent cancer will of course vary depending upon the nature and severity of the disorder, the age and condition of the subject, and other factors readily determined by one of ordinary skill in the art.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The references cited throughout this application are herein incorporated by reference.

The documents mentioned herein are incorporated herein by reference.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

### EXAMPLES

### Materials and Methods

Cell Culture and Conditioned Media Collection

Human pancreatic adenocarcinoma cell line BxPC3 (American Type Culture Collection Rockville, Md.) was incubated in RPMI 1640 (Gibco) supplemented with 10% fetal calf serum (Gibco), 100 μg/ml penicillin G, and 100 mg/ml streptomycin (Pharmacia, Sweden). Cells were maintained on T-75 tissue culture flask (Falcon) and grown in 5% CO₂/95% air at 37° C. in an humidified incubator. Conditioned media was produced by adding of RPMI 1640/5% FCS (100 ml) to near confluent BxPc3 cells in 900 cm² roller bottles (Corning), and incubating for 72 hr. Conditioned media was collected, filtered (0.45 μm), and stored at 4° C. Serum free conditioned medium was generated by the addition of the corresponding serum free media to 90-95% confluent cells after washing twice with PBS. After 24 hr of incubation, the serum free conditioned media was collected, filtered and used immediately.

BCE Assays

Bovine capillary endothelial cells were maintained in DMEM with 10% heat-inactivated BCS, antibiotics, and 3 ng/mL recombinant human bFGF (Scios Nova, Calif.). Cells were washed with PBS and dispersed in a 0.05% solution of trypsin. A cell suspension was made with DMEM/10% BCS/1% antibiotics and the concentration adjusted to 25,000 cells/ml. Cells were plated onto gelatinized 24-well culture plates (0.5 mL/well) and were incubated at 37° C. in 10% CO₂ for 24 h. The media was replaced with 0.25 mL of DMEM/5% BCS/1% antibiotics, and the test sample applied. After 20 min incubation, media and bFGF were added to each well to obtain a final volume of 0.5 mL DMEM/5% BCS/1% antibiotics/1 ng/mL bFGF. After 72 h, cells were dispersed in trypsin, resuspended in Isoton II (Fisher scientific, Pa.) and counted by Coulter counter.

Purification of Human and Bovine DBP

Human and bovine DBP were purified form respective sera using 25-hydroxyvitamin D₃ affinity chromatography as described (29) with suitable modifications. Human or bovine sera was diluted with equal volume of column buffer (50 mM Tris HCl, pH 8.3, 150 nM NaCl, 1.5 mM EDTA) and applied to the 25-hydroxyvitamin D₃ affinity column at 4° C. The column was washed with the column buffer (20 times the bed volume) and bound proteins eluted with 1M acetate buffer pH 5.0. The salts were dialyzed away and proteins were further fractionated on an hydroxyapatite column equilibrated with 10 mM potassium phosphate buffer, pH 7.0. The column was washed with excess equilibration buffer and bound DBP eluted with 75 mM potassium phosphate buffer, pH 7.0.

Purification of DBP-maf from BxPC3 Conditioned Media

BxPC3 conditioned media was applied to a 25-hydroxyvitamin D₃ affinity column (equilibrated with 50 mM Tris HCl, pH 8.3 buffer at 4° C.). The column was washed with RPMI 1640 followed by 50 mM Tris HCl, pH 8.3 buffer. Bound DBP/DBP-maf was eluted with 1M acetate buffer, pH 5.0. The proteins were further purified using hydroxyapatite chromatography as described above.

Generation of DBP-maf from DBP

Sialidase and βgalactosidase (2 Units each) were coupled to 0.6 g of CNBr activated Sepharose (Pharmacia) according to manufacturer's instructions. The immobilized enzymes were stored at 4° C. after a final wash with coupling buffer until needed.

DBP (100 μg) was incubated with a mixture of immobilized sialidase and β-galactosidase (0.2 units of activity each) in PBS-Mg (10 mM sodium phosphate buffer pH 5.5, 0.9% sodium chloride and 1 mM MgSO₄) at 37° C. on an end-to-end shaker for 2 hours. The gel was sedimented by centrifugation at 600×g at 4° C. for 5 minutes. The supernatant (containing DBP-maf/25-OH-D₃-DBP-maf) was collected and sterilized by filtering through 0.22 micron filter (10)⁷. Possible endotoxin contamination of the DBP-maf preparations was eliminated by removing endotoxin using Detoxi Gel™ (Pierce).

Chick Chorioallantoic Membrane (CAM) Assay.

Chick embryos were prepared as reported. Sample dissolved in matrigel (10 μL) was micropipetted onto the outer third of the chorioallantoic membrane of day 6 embryos. This procedure was accomplished by using a 3×-6× stereoscope. Antiangiogenic activity was measured by the diameter of the avascular zone around the sample between 48 and 96 hours. No zone was scored as “−”, a zone with diameter of 2 mm beyond the edge of the mesh was scored as “+”, with each 2 mm increment being scored as an additional “+”. CAM assays were scored by an investigator who was blinded to the identity of the samples.

Animal Studies.

All animal work was performed in the animal facility at Children's Hospital, Boston in accordance with federal, local, and institutional guidelines. Male 6-8 weeks old immuno-compromised mice (SCID, Mass General Hospital, Boston, Mass.) or C57BL6/J (Jackson Labs, Bar Harbor, Me.) mice were acclimated, caged in groups of four or less in a barrier care facility and their backs were shaved. All mice were fed a diet of animal chow and water ad libitum. Animals were anesthetized with methoxyflurane (Pittman-Moore Inc., Mundelein, Ill.) before all procedures and observed until fully recovered. Animals were sacrificed by CO₂ asphyxiation.

Mouse Tumor Model

Preparation of Tumor Cells for Implantation in Mice

Pancreatic cancers cells and HT 1080 cells grown in cell culture as described above, were washed with PBS, dispersed in a 0.05% solution of trypsin, and resuspended. After centrifugation (4000 rpm for 10 min at room temperature), the cell pellet was resuspended in RPMI and the concentration was adjusted to 12.5×10⁶ cells/ml. After the site was cleaned with ethanol and tumor cells were s.c. injected with 2.5×10⁶ cells in 0.2 ml RPMI.

Preparation of Lewis Lung Cells for Implantation in Mice

Animals with Lewis lung carcinoma tumors of 600-1200 mm³ were sacrificed, and the skin overlying the tumor was cleaned with betadyne and ethanol. In a laminar flow hood, tumor tissue was excised under aseptic conditions. A suspension of tumor cells in 0.9% normal saline was made by passage of viable tumor tissue through a sieve and a series of sequentially smaller hypodermic needles of diameter 22- to 30-gauge. The final concentration was adjusted to 1×10⁷ cells/ml and the suspension was placed on ice. After the site was cleaned with ethanol, mice were injected with 2.5×10⁶ cells in 0.2 ml PBS.

Double Side Tumor Model

A primary tumor of pancreatic cancer cells was generated by injecting 2.5×10⁶ cells (0.2 ml) in the left flank. Tumors were measured with a dial-caliper, and volumes were determined using the formula width²×length×0.52. When the tumor volume was at least 400-500 mm³ (2-2.5% of body weight), which occurred within 14-21 days, the secondary tumor was transplanted in the contra-lateral flank (2.5×10⁶ cells in 0.2 ml). Control mice received an identical injection of secondary tumor cells at the same time. Tumors were measured every third day. The suppression rate induced by the primary tumor was seen as delayed, reduced, or null growth of the secondary tumor, with respect to the controls. Alternatively, pancreatic cancer cells were injected in both flanks (2.5×10⁶ cells in 0.2 ml). Tumors were measured routinely every third day. Male mice, 6-8 weeks of age, received s.c injections of 4×10⁶ tumor cells suspended in 100 μl of RPMI in the proximal dorsum. For further in vivo passages, s.c. tumors were resected under aseptic conditions, minced, and re-implanted s.c into new animals. Human pancreatic cancer, and HT1080 or Lewis Lung carcinoma were inoculated into the subcutaneous mid-dorsum of SCID or C57BL6/J mice.

Histology

Specimens were fixed in 10% neutral buffered formalin at 4° C. overnight and embedded in paraffin. Five-micron sections were made and routinely stained with hematoxylin and eosin. To visualize endothelial cells, sections were incubated in rat antiserum against mouse cd31 (pecam; 1:250; PharMingen, San Diego) overnight at 4° C. A secondary antibody, biotinylated rabbit anti-rat, mouse-adsorbed (1:200, Vector, Burlingame, Calif.) was followed by streptavidin-horseradish peroxidase and tyrarnide signal amplification (New England Nuclear, Boston). AEC (Dako, Carpenteria, Calif.) and Gill's hematoxylin were the chromagen and counterstain. Cells of hematopoietic origin such as macrophages were immunostained with rat anti-mouse cd45 antiserum (leukocyte common antigen, Ly-5′1:100, PharMingen). After the same secondary antibody as above, avidin/biotin conjugated to alkaline phosphatase (Vector) was used with a New Fuschin chromagen (BioGenex, San Ramon, Calif.).

### Results

Purification of Anti-Endothelial DBP-MAF from Pancreatic Cancer Cell Conditioned Media

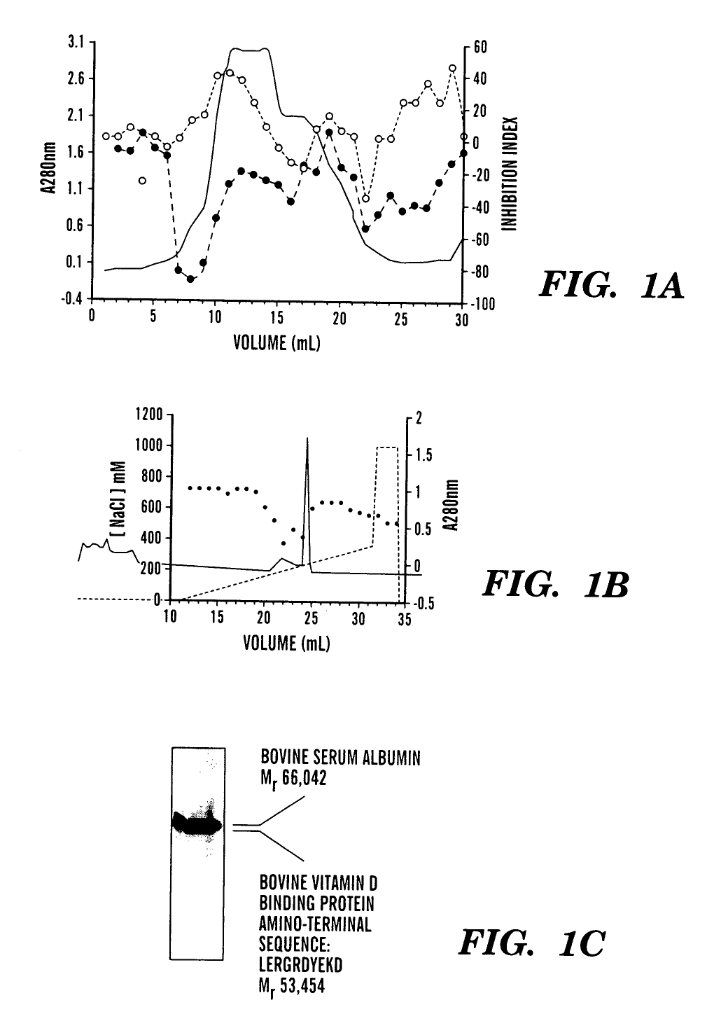

The pancreatic cancer cell, BxPC3, was able to inhibit the growth of a secondary tumor implant by up to 80% (Table 1), as determined in an in vivo model in extensive use in our laboratory. A different pancreatic cancer cell line (ASPC-1) was unable to inhibit the growth of secondary tumors, suggesting that BxPC3 was producing an antiangiogenic factor. However, the conditioned media obtained from BxPC3 cells was unable to inhibit the proliferation of endothelial cells (data not shown). This was possibly due to the relatively high concentrations of growth factors such as bFGF (6-8 pg/mL) and VEGF (3000-6000 pg/mL) present in the conditioned media after 24-48 hours. These proangiogenic cytokines bind heparin (5). We therefore fractionated the conditioned media on heparin-Sepharose. Material that did not bind to heparin-Sepharose exhibited anti-endothelial activity, as determined using an endothelial cell proliferation assay. This activity was further purified using a combination of Q-Sepharose and monoQ anion exchange chromatography (see Materials and Methods). Anti-endothelial fractions eluting from the monoQ column exhibited two bands by SDS-PAGE (FIG. 1C). These two proteins were resolved using C4 RP-HPLC. Mass spectrometry and sequence analysis indicated that one of the proteins (Mr 66,042) was BSA, while the other protein (Mr 53,454) had the amino terminal sequence LERGRDYEKD. This sequence is 90% similar to the amino terminal sequence of human vitamin D binding protein (DBP). We assigned an identity of bovine DBP to our anti-endothelial protein.

Selective Deglycosylation Converts DBP to an Antiangiogenic Factor

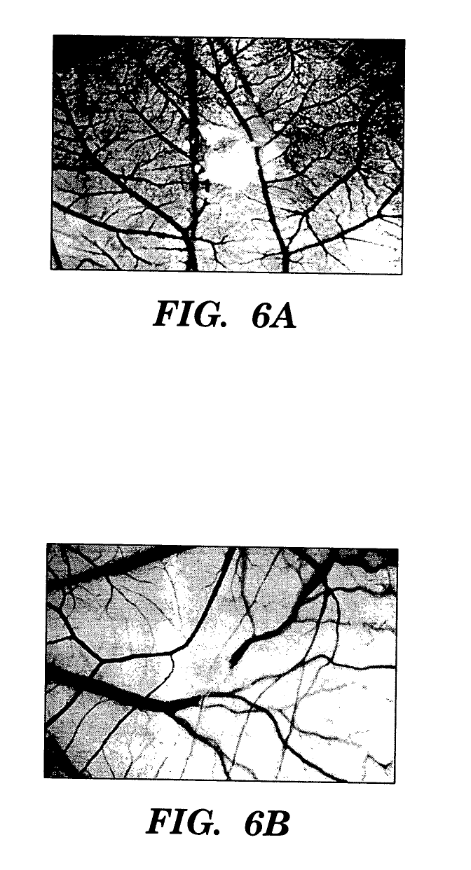

Human DBP, expressed in an E. coli system, was unable to inhibit angiogenesis in the CAM (Table 2). Thus, we hypothesized that the BxPC-3 cells were modifying bovine DBP in some way to generate an anti-endothelial protein. A known biochemical modification of DBP is deglycosylation to produce DBP-maf. Furthermore, it is well known that cancer cells produce glycosidases. BxPC-3 cells produce sialidase and β-galactosidase (Table 3). In order to test this hypothesis, we purified human DBP, deglycosylated it using immobilized β-galactosidase and sialidase as described (see Materials and Methods) and tested this DBP-maf in the CAM assay. DBP-maf was able to inhibit angiogenesis in the CAM assay (Table 2 and FIGS. 6A and 6B).

DBP Purified from BXPC3 Conditioned Media can Activate Macrophages in a Similar Manner to Human and Bovine DBP-MAF

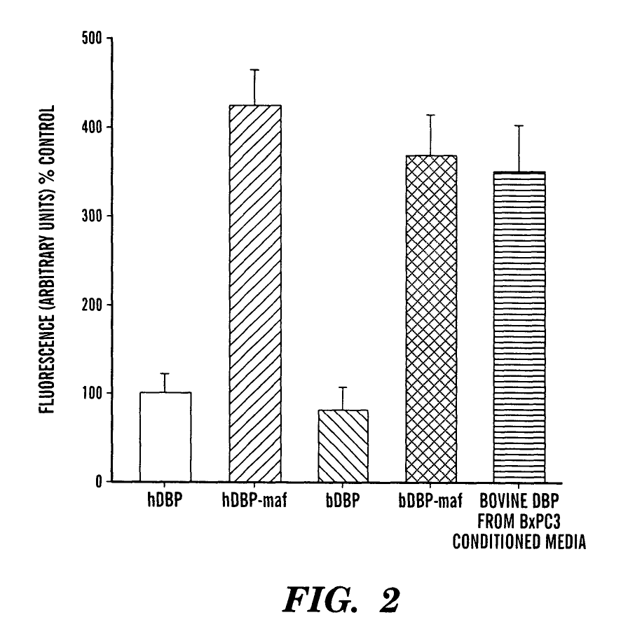

Human and bovine DBP were purified as described, and enzymatically converted to DBP-maf These DBP-maf proteins were able to activate macrophages (FIG. 2) as determined by a superoxide generation assay (see Materials and Methods). The increase in superoxide generation by macrophages exposed to DBP-maf was 3-4 fold. DBP purified from BxPC3 conditioned media was able to activate macrophages, with a similar increase in superoxide production of 3-4 fold over bovine DBP (FIG. 2). These data, the inhibition of endothelial cell proliferation, and the activation of macrophages, indicate that the DBP purified from BxPC3 conditioned media, has been converted to the DBP-maf form.

Systemically Administered DBP-MAF is Able to Inhibit the Growth of Solid Tumors.

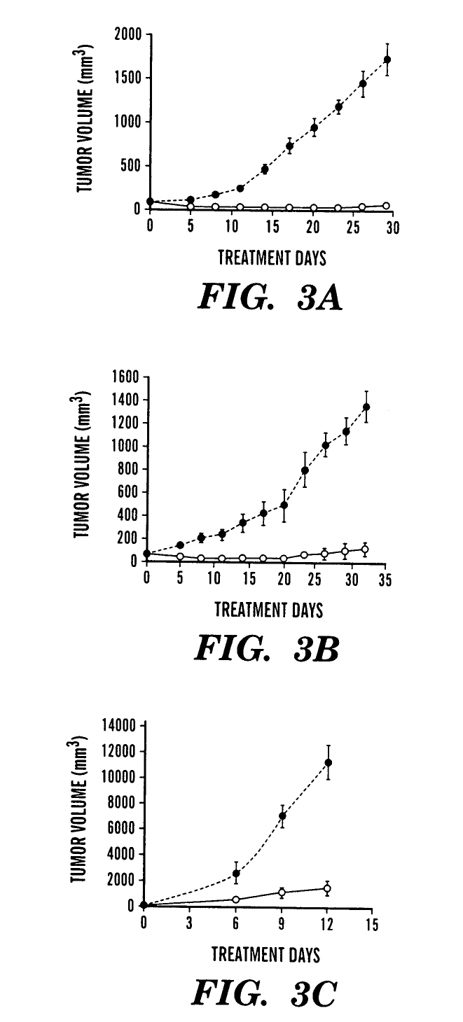

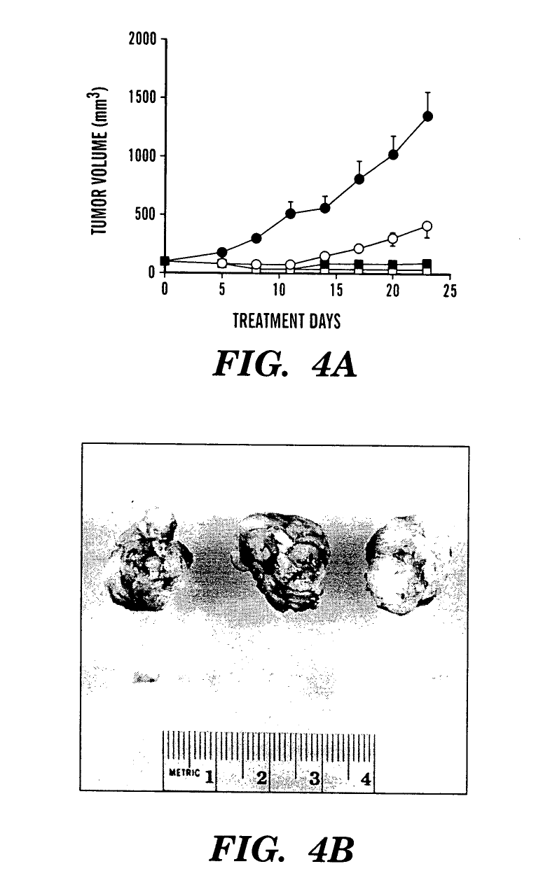

Tumors were implanted on the flank of a mouse, and when the tumor had attained a volume of 100 mm³ therapy was started. Control animals received saline or E. coli expressed human vitamin D binding protein and treated animals received a sub-cutaneous injection of DBP-maf (4 ng/Kg/day) for 28-30 days. The growth of human pancreatic tumors (BxPC3 and SU88.86) in immune compromised mice were inhibited dramatically (FIGS. 3A-C), with essentially no growth of the treated tumor observed over the course of the experiment. The growth of Lewis lung carcinoma in a normal C57BL6/J mouse was also markedly inhibited by DBP-maf at this dose, although there was a measurable increase in the volume of the treated tumor over the course of the experiment. We observed a dose response to the therapy when we treated BxPC3 tumors (FIGS. 4A-B). Animals receiving human DBP expressed in E coli exhibited tumor growth, whereas animals receiving the maximum dose in this experiment (4 μg/Kg/day) showed no growth of established tumors, with evidence of regression of established tumors. Animals receiving 4 ng/Kg/day also exhibited little or no tumor growth even after 24 days on therapy, whereas animals receiving 4 ng/Kg every 4 days displayed some growth in established tumors (FIGS. 4A-B).

Histology Results.

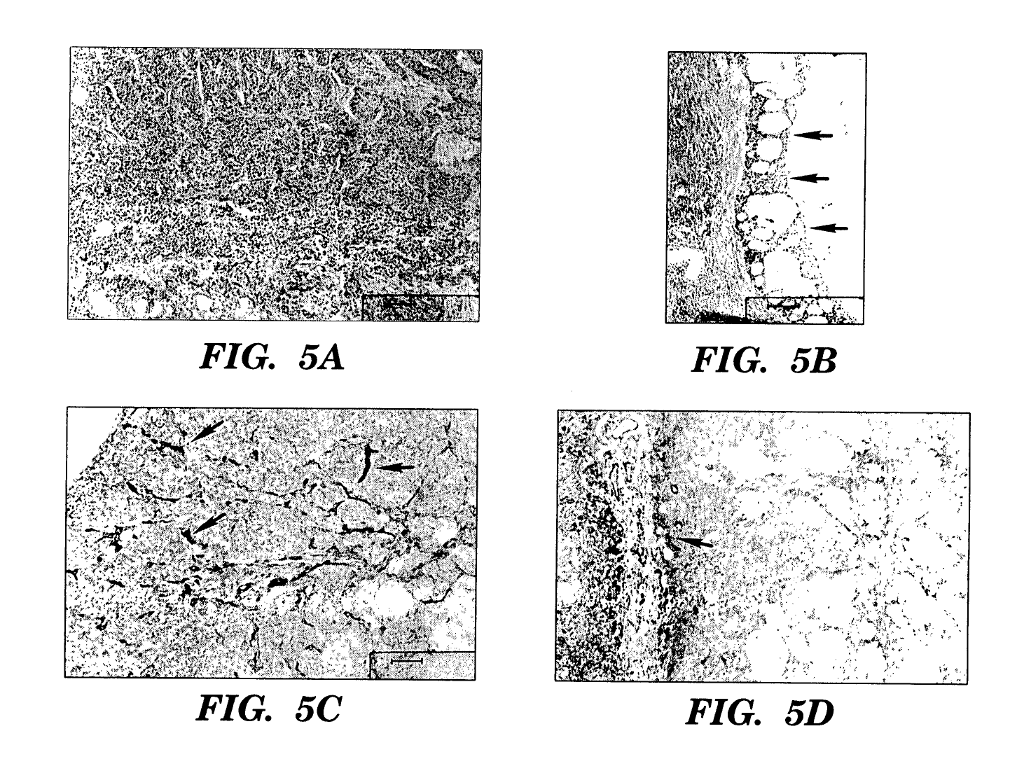

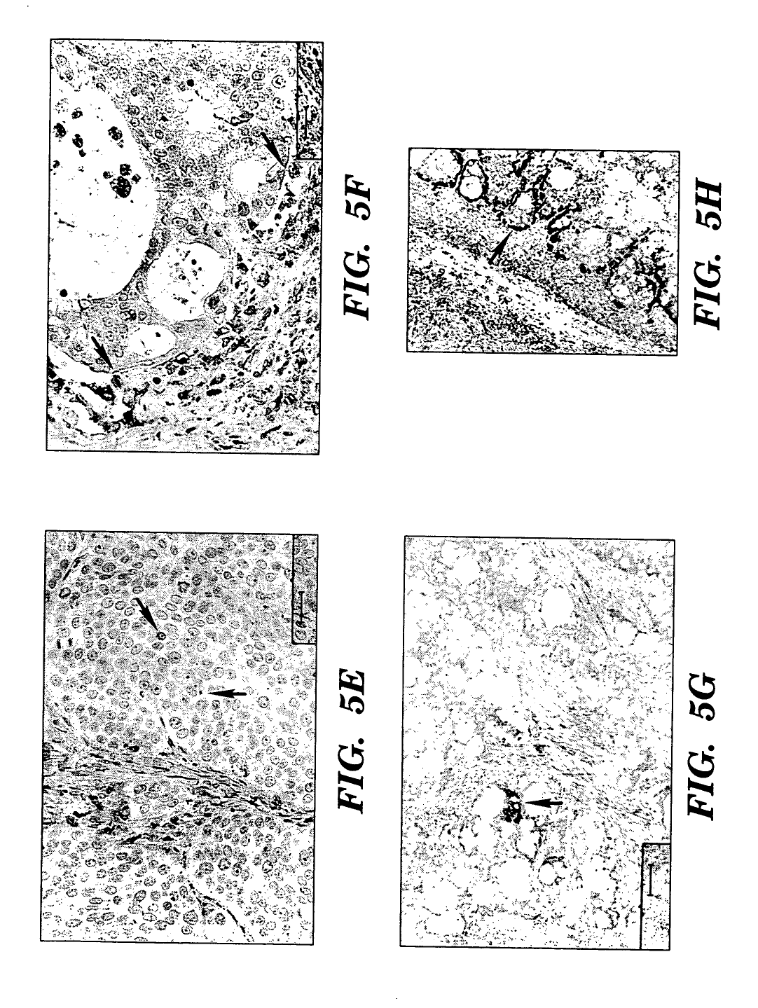

Histological analysis showed that treated tumors were comprised mainly of a small necrotic cyst with a layer of viable tumor cells at the outer perimeter, adjacent to a layer of healthy connective tissue (FIG. 5B, arrowed). Untreated tumors (FIG. 5A) showed no such morphology, instead comprising a mass of viable tumor cells. Immunostaining for the endothelial cell specific marker CD31 revealed that untreated tumors had a higher microvessel density (FIG. 5C, arrowed) compared to treated tumors (FIG. 5D, arrowed). Note that the adjacent layer of connective tissue appears to be vascularized in treated tumors (FIG. 5D). Furthermore, immunostaining directed against the macrophage specific marker Mac-3 revealed that treated tumors had been infiltrated by macrophages (FIG. 5F, arrowed), whereas control tumors had much reduced staining for this antigen (FIG. 5E). Staining for another macrophage marker, Leucocyte Common Antigen (LCA), which will also stain NK-cells, also showed infiltration of the treated tumor by cells of leucocyte origin (FIGS. 5G and 5H).

Discussion

We have purified an anti-endothelial form of DBP from the conditioned media of a human pancreatic cancer cell line. The anti-endothelial form of the protein is converted from systemic DBP by selective deglycosylation of the O-linked oligosaccharide in the carboxy terminal domain of the protein. The pancreatic cells are producing the glycosidases required for this conversion. This explains why a human pancreatic cancer, under tissue culture conditions in the presence of bovine serum, is producing a bovine form of DBP-maf.

This anti-endothelial form of DBP-maf can directly inhibit the proliferation of endothelial cells, and inhibit angiogenesis in the CAM assay. DBP-maf has no effect on the proliferation of human pancreatic cancer cells (BxPC3) or smooth muscle cells, suggesting endothelial cell specificity. DBP-maf, generated from purified human DBP, was effective as an anti-tumorigenic drug. Administration of low daily doses (4 ng/Kg) of DBP-maf caused marked inhibition of solid tumor growth in murine models. Further, DBP-maf was able to regress established pancreatic tumors in immune compromised mice when administered at higher doses. Although administration of human DBP-maf caused inhibition of growth of a lewis lung carcinoma in C57BL6/J mice, the inhibition was not as marked as treatment of human pancreatic cancer in immune compromised mice. This observation may have two explanations: the lewis lung carcinoma is a faster growing tumor that requires a more aggressive treatment regimen, or the C57BL6/J mice are mounting an immune response against a human protein which has been shown to be a potent adjuvant for immunization (14).

DBP-maf has been used successfully to treat Ehrlich ascites tumor in a mouse model (11, 12). Treatment of mice with low doses of DBP-maf (100 pg/mouse) resulted in significantly increased survival times as compared to control mice (11). Further, treatment with DBP-maf eradicated the tumor, as measured by peritoneal counts of tumor cells at the end of treatment (12). Although DBP-maf showed no efficacy against a squamous cell carcinoma in a murine model when administered alone (13), it proved to be curative when administered as an adjuvant to photodynamic therapy.

This selectively deglycosylated form of the protein also activates macrophages (15). We have also shown that DBP-maf, either generated enzymatically or purified from BxPC3 conditioned media, can activate macrophages. Macrophages are terminally differentiated cells that produce a number of potent angiogenic cytokines and growth factors, as well as ECM degrading enzymes. Thus macrophages can influence various stages of angiogenesis either positively or negatively (16). There are also differences in the angiogenic potential of classically versus alternatively activated macrophages (17). Alternatively activated macrophage-rich lesions tend to be highly vascularized, whereas classically activated macrophage-rich lesions tend not to be ¹⁴.

The activated macrophages may secrete an antiangiogenic factor that contributes to the potency of this molecule. The identity of this factor is unknown at present, although efforts are underway to assign an identity to it. We assayed the DBP-maf stimulated macrophage conditioned media for cytokines known to have a role in angiogenesis. DBP-maf did not induce the secretion of IFNg or IL-12, both of which were undetectable by ELISA in the macrophage conditioned media (data not shown). The absence of IFNg in the macrophage conditioned media argues against the direct involvement of this cytokine and also any involvement of IL-18. IL-18 is reported to be antiangiogenic and anti-tumorigenic. However, it is a potent IFNg-inducing cytokine, thus one would expect to find IFNg if IL-18 was involved. IL-12 has also been shown to be a potent antiangiogenic cytokine which again mediates its effect by inducing IFNg. We have directly demonstrated the absence of IL-12 in the conditioned media of macrophages exposed to DBP-maf.

It has been hypothesized that DBP-maf activates macrophages as the first step in an immune developmental process involving macrophages acting as antigen presentation cells, and lymphocytes generating antibodies against tumor antigens (13, 21). It has been suggested that the effectiveness of multiple administration of DBP-maf against Ehrlich ascites tumor is due to a developed immunity against the tumor (11). In support of this concept, Ehrlich ascites tumor cells cannot grow in mice previously immunized with killed Ehrlich asciites (22). It is also known that the tumoricidal capacity of macrophages is observed preferentially through the IgG (Fc-receptor)-mediated pathway (23, 24, 25) and DBP-maf is a potent adjuvant in immunization for antibody production. However, we have observed antitumorigenic activity with DBP-maf therapy in immune compromised mice, deficient in B- and T-lymphocytes. Such an observation argues against a wholly immune mediated mechanism. Further, we saw no anti-proliferative effect against pancreatic cancer cells by either DBP-maf itself, or the conditioned media of DBP-maf stimulated macrophages. These observations argue for another mechanism, which we hypothesize to be antiangiogenic, based on the observed anti-endothelial activity and antiangiogenic activity of DBP-maf and conditioned media from DBP-maf stimulated macrophages.

It has been noted that cancers can secrete endo and exoglycosidases (26, 27, 28). Evidence has been presented showing that cancerous cells secrete an I-N-acetylgalactosaminidase which cleaves the entire O-linked oligosaccharide from DBP. Such a deglycosylation renders the molecule incapable of being converted to DBP-maf. It has been speculated that this may be a mechanism whereby a cancer can evade an inflammatory response (22). In fact, the presence of I-N-acetylgalactosaminidase activity in the bloodstream of a cancer patient can serve as a prognostic index for the disease (22). It is not surprising then that a cancer cell may also be able to secrete the enzymes responsible for efficient conversion of DBP to DBP-maf. Thus, BxPC3, in secreting glycosidases capable of generating DBP-maf from DBP may be hoist with it's own petard.

In conclusion, a human pancreatic cancer cell line is capable of generating DBP-maf from DBP. This molecule has inherent anti-endothelial activity. Systemic administration of DBP-maf can inhibit the rate of tumor growth of various solid tumors, and can, in some cases, cause regression of established tumors. Such tumor inhibition and regression was observed in immunecompromised mice, arguing against a wholly immune response mediated mechanism. We believe that DBP-maf is working, at least in part, through an antiangiogenic mechanism, and that this antiangiogenic mechanism is amplified by a powerful unknown antiangiogenic factor secreted by macrophages. Our evidence for this hypothesis comes from in vitro endothelial cell proliferation assays, and CAM assays. We are currently trying to purify and characterize the unknown factor secreted by macrophages, although it does not appear to be IL-12, IFNγ, or TNFα. DBP-maf has efficacy as an anti-tumorigenic therapy at relatively low doses in mice, compared to other antiangiogenic therapies (1, 2).

TABLE 1
Inhibition of secondary tumour growth
by a primary Pancreatic Cancer tumour
in an in vivo mouse model.

| | Secondary Tumor | | |
|---|---|---|---|
| Primary Tumor | Lewis Lung | HT1080 | BxPC3 |
| BxPC3 | 84% | 73% | 81% |
| ASPC-1 | 10% | 7% | Not Determined |

Tumor cells were prepared and implanted into SCID mice as described. At the end of the experiment, tumor volume was measured. The volume of an inhibited tumor was compared to the volume of an uninhibited control tumor, and the ratio expressed as a percentage.

TABLE 2
Inhibition of angiogenesis in the CAM assay by DBP-maf.

| | CAM Response |
|---|---|
| E coli expressed DBP | − |
| 0.5 μg | |
| E coli expressed DBP | − |
| 5 μg | |
| DBP-maf-0.5 μg | −/+ |
| DBP-maf 5 μg | +++ |

The CAM assay was performed and graded as described in Materials and Methods.

TABLE 3
BxPC3 cells express sialidase and galactosidase activity

| | | Galactosidase |
|---|---|---|
| | Sialidase activity | activity |
| Cancer cell line | (fluorescence units) | (fluorescence units) |
| BxPC3 | 40750 + 353 | 8570 + 86 |
| ASPC-1 | ND | ND |

Human pancreatic cancer cells were assayed for the presence of sialidase and β-galactosidase as described. Activities are expressed as arbitrary fluorescence units.

The following references are cited throughout the specification and are incorporated herein by reference.

1. O'Reilly M S, Holmgren L, Chen C, Folkman J (1996) Angiostatin induces and sustains dormancy of human primary tumors in mice. Nat Med 2 689-92

2. Boehm T, Folkman J, Browder T, O'Reilly M S (1997) Antiangiogenic therapy of experimental cancer does not induce acquired drug resistance. Nature 390 404-7

3. Swamy N. Roy A. Chang R. Brisson M. Ray R. (1995) Affinity purification of human plasma vitamin D-binding protein. Protein Express.Purif. 6:185-188.

4. Folkman J (1995) Angiogenesis in cancer, vascular, rheumatoid and other disease. Nat Med. 1:27-31

5. O'Reilly M S, Holmgren L, Shing Y, Chen C, Rosenthal R A, Moses M, Lane W S, Cao Y, Sage E H, Folkman J Angiostatin: a novel angiogenesis inhibitor that mediates the suppression of metastases by a Lewis lung carcinoma. (1994) Cell 79 315-28

6. O'Reilly M S, Boehm T, Shing Y, Fukai N, Vasios G, Lane W S, Flynn E, Birkhead J R, Olsen B R, Folkman J (1997) Endostatin: an endogenous inhibitor of angiogenesis and tumor growth. Cell 88 277-85

7. O'Reilly M S, Pirie-Shepherd S, Lane W S, Folkman J (1999) Antiangiogenic activity of the cleaved conformation of the serpin antithrombin. Science 285 1926-8

8. O'Reilly, M. S., Rosenthal, R., Sage, E. H., Smith, S., Holmgren, L., Moses, M., Shing, Y., and Folkman, J. The suppression of tumor metastases by a primary tumor. (1993) Surg. Forum 44 474-476

9. Ray R (1996) Molecular recognition in vitamin D binding protein. Proc Soc Exp Biol Med 212 305-12.

10. Yamamoto N, Kumashiro R (1993) Conversion of vitamin D3 binding protein (group-specific component) to a macrophage activating factor by the stepwise action of beta-galactosidase of B cells and sialidase of T cells. J Immunol 151 2794-802.

11. Yamamoto N, Naraparaju V R (1997) Immunotherapy of BALB/c mice bearing Ehrlich ascites tumor with vitamin D-binding protein-derived macrophage activating factor. Cancer Res 57 2187-92.

12. Koga Y, Naraparaju V R, Yamamoto N (1999) Antitumor effect of vitamin D-binding protein-derived macrophage activating factor on Ehrlich ascites tumor-bearing mice. Proc Soc Exp Biol Med 220 20-6.

13. Korbelik M, Naraparaju V R, Yamamoto N (1997) Macrophage-directed immunotherapy as adjuvant to photodynamic therapy of cancer. Br J Cancer 75 202-7.

14. Yamamoto N, Naraparaju V R (1998) Structurally well-defined macrophage activating factor derived from vitamin D3-binding protein has a potent adjuvant activity for immunization. Immunol Cell Biol 76 237-44.

15. Yamamoto, N., and Homma S (1991) Vitamin D3 binding protein (group-specific component) is a precursor for the macrophage-activating signal factor from lysophosphatidylcholine-treated lymphocytes. Proc Natl Acad Sci USA 88 8539-43

16. Ono M, Torisu H, Fukushi J, Nishie A, Kuwano M (1999) Biological implications of macrophage infiltration in human tumor angiogenesis. Cancer Chemother Pharmacol 43 suppl 69-71

17. Kodelja V, Muller C, Tenorio S, Schebesch C, Orfanos C E, Goerdt S (1997) Differences in angiogenic potential of classically vs alternatively activated macrophages. Immunobiology 197 478-93.

18. Cao R, Farnebo J, Kurimoto M, Cao Y (1999) Interleukin-18 acts as an angiogenesis and tumor suppressor. FASEB J 13 2195-202

19. Voest E E, Kenyon B M, O'Reilly M S, Truitt G, D'Amato R J, Folkman J (1995) Inhibition of angiogenesis in vivo by interleukin 12. J Natl Cancer Inst 87 581-6.

20. Frater-Schroder M, Risau W, Hallmann R, Gautschi P, Bohlen P (1987) Tumor necrosis factor type alpha, a potent inhibitor of endothelial cell growth in vitro, is angiogenic in vivo. Proc Natl Acad Sci USA 1987 84 5277-81

21. Yamamoto N, Naraparaju V R, Asbell S O (1996) Deglycosylation of serum vitamin D3-binding protein leads to immunosuppression in cancer patients. Cancer Res 56 2827-31

22. Morton D, Eilber F R, Malmgren R A, Wood W C (1970) Immunological factors which influence response to immunotherapy in malignant melanoma. Surgery 1970 68 158-63

23. Yamamoto, N., Hoober, J. K., Yamamoto, S., and Yamamoto, N., (1992) Tumoricidal capacities of macrophages photodynamically activated with hematoporphoryin derivative. Photochem. Photobiol. 56 245-250

24. Weiner L M, Moldofsky P J, Gatenby R A, O'Dwyer J, O'Brien J, Litwin S, Comis R L (1980) Antibody delivery and effector cell activation in a phase II trial of recombinant gamma-interferon and the murine monoclonal antibody CO17-1A in advanced colorectal carcinoma. Cancer Res 48 2568-73

25. Houghton A N, Mintzer D, Cordon-Cardo C, Welt S, Fliegel B, Vadhan S, Carswell E, Melamed M R, Oettgen H F, Old L J (1985) Mouse monoclonal IgG3 antibody detecting GD3 ganglioside: a phase I trial in patients with malignant melanoma. Proc Natl Acad Sci USA 82 1242-6

26. Yamamoto, N., Naraparaju, V. R., Srinivasula, S. M., (1995) Structural modification of serum vitamin-D₃ binding protein and immunosuppresion in HIV-infected patients AIDS Res. Hum. Retroviruses 11 1373-1378

27. Yang F, Bergeron J M, Linehan L A, Lalley P A, Sakaguchi A Y, Bowman B H (1990) Mapping and conservation of the group-specific component gene in mouse. Genomics 7 509-16.

28. Yamamoto N, Lindsay D D, Naraparaju V R, Ireland R A, Popoff S N (1994) A defect in the inflammation-primed macrophage-activation cascade in osteopetrotic rats. J Immunol 152 5100-7.

29. Swamy N. Roy A. Chang R. Brisson M. Ray R. (1995) Affinity purification of human plasma vitamin D-binding protein. Protein Express.Purif. 6:185-188.

30. Frater-Schroder M, Risau W, Hallmann R, Gautschi P, Bohlen P (1987) Tumor necrosis factor type alpha, a potent inhibitor of endothelial cell growth in vitro, is angiogenic in vivo. Proc Natl Acad Sci USA 1987 84 5277-81.

31. Folkman and Shing, J. Biol. Chem. 267 (16), 10931-10934 (1992).

32. Folkman, N. Engl. J. Med., 285:1182-1186 (1971).

33. Yanagisawa-Miwa, et al., Science, 257:1401-1403 (1992).

34. Baffour, et al., J Vasc Surg, 16:181-91 (1992).

35. Pu, et al., J Surg Res, 54:575-83 (1993).

36. Takeshita, et al., Circulation, 90:228-234 (1994).

37. Takeshita, et al., J Clin Invest, 93:662-70 (1994).

38. Battegay, J. Mol. Med., 73, 333-346 (1995).

39. Hanahan et al., Cell, 86, 353-364 (1996).

40. Folkman, N. Engl. J. Med., 333, 1757-1763 (1995).

Although the foregoing invention has been described in some detail by way of illustration and example for the purposes of clarity of understanding, one skilled in the art will easily ascertain that certain changes and modifications may be practiced without departing from the spirit and scope of the appended claims.

What is claimed is:

1. A method of inhibiting angiogenesis in a mammal having an angiogenic disease comprising pulsed or sustained release administration to said mammal of an effective antiangiogenic amount of deglycosylated Vitamin D binding protein (DBP-maf).

2. The method of claim 1, wherein the interval between pulses is 24 hours or greater.

3. The method of claim 2, wherein the plurality of pulses comprises from about 5 to about 10 pulses.

4. The method of claim 2, wherein the plurality of pulses comprises greater than 20 pulses.

5. The method of claim 2, wherein the interval is from 1 to about 7 days.

6. A method of inhibiting angiogenesis at a site in a mammal having an angiogenic disease other than cancer comprising administration of an antiangiogenic effective amount of DBP-maf.

7. A method of inhibiting an inducer of angiogenesis at a site in a mammal having an angiogenic disease other than cancer comprising administration of an antiangiogenic effective amount of DBP-maf.

8. A method of inhibiting angiogenesis at a tumor site in an immunocompromised mammal comprising administration of an antiangiogenic effective amount of DBP-maf.

9. The method of claim 8, wherein the immunocompromised mammal is immunocompromised due to a T or B lymphocyte deficiency.

10. A method of inhibiting angiogenesis in a mammal having angiogenic disease comprising administration of an antiangiogenic effective amount of DBP-maf and a second anti-angiogenic factor, and a pharmaceutically acceptable carrier.

11. A method of inhibiting angiogenesis at a site in a mammal having an angiogenic disease, said angiogenic disease selected from the group consisting of diabetic retinopathy, retrolental fibroplasia, trachoma, neovascular glaucoma, psoriases, immune-inflammation, non-immune inflammation, atherosclerosis, and excessive wound repair, comprising administration of an antiangiogenic effective amount of DBP-maf.

12. A method of inhibiting angiogenesis at a site in a mammal having an angiogenic disease, said angiogenic disease consisting of immune inflammation wherein the immune inflammation is caused by an autoimmune disease selected from the group consisting of rheumatoid arthritis, systemic lupus erythematosus, thyroiditis, Goodpasture's syndrome, systemic vasculitis, scleroderma, Sjogren's syndrome, sarcoidosis, and primary biliary cirrhosis and said method comprising administration of an antiangiogenic effective amount of DBP-maf.

13. A method of inhibiting angiogenesis according to claim 7 wherein the site is a dermis, epidermis, endometrium, retina, surgical wound, gastrointestinal tract, umbilical cord, liver, kidney, reproductive system, lymphoid system, central nervous system, breast tissue, urinary tract, circulatory system, bone, muscle, or respiratory tract.

14. A method according to claim 7 wherein the inducer of angiogenesis is selected from the group consisting of basic fibroblast growth factor, acidic fibroblast growth factor, hepatocyte growth factor, IL-8 and vascular endothelial growth factor.

15. A method according to claim 6 wherein the site is a dermis, epidermis, endometrium, retina, surgical wound, gastrointestinal tract, umbilical cord, liver, kidney, reproductive system, lymphoid system, central nervous system, breast tissue, urinary tract, circulatory system, bone, muscle or respiratory tract.

16. A method according to claim 6 further comprising administration of an effective amount of a second anti-angiogenic agent.

17. A method of inhibiting angiogenesis according to claim 10, wherein the tumor site is a Kaposi's sarcoma.

18. A method of inhibiting angiogenesis at a tumor site according to claim 8, wherein the T or B cell deficiency is congenital or acquired.

19. A composition comprising deglycosylated Vitamin D binding protein (DPB-MAF) and 1,25(OH)₂ Vitamin D or a derivative thereof.

20. A method for treating a hormone dependent cancer in a host in need thereof, said method comprising administering to said host an effective amount of a composition comprising deglycosylated Vitamin D binding protein (DBP-maf) and 1,25(OH)₂ Vitamin D or a derivative thereof.

21. The method of claim 20, wherein the hormone dependent cancer is prostate or breast.
